(19) United States
(12) Patent Application Publication
(10) Pub. No.: US 2026/0338879 A1
(43) Pub. Date: Oct. 29, 2026

(54) MICROMECHANICAL RESONATORS

(71) Applicant: Northern Digital Inc., Waterloo (CA)

(72) Inventors: Gereon Vogtmeier, Aachen (DE); Bernhard Gleich, Hamburg (DE); Peter Cäsar Mazurkewitz, Hamburg (DE); Christian Jörg Schilling, Thayngen (CH)

(21) Appl. No.: 19/276,334

(22) Filed: Jul. 22, 2025

Related U.S. Application Data
(60) Provisional application No. 63/793,237, filed on Apr. 23, 2025.
(60) Provisional application No. 63/793,271, filed on Apr. 23, 2025.

(51) Int. Cl. G01R 33/00

(52) U.S. Cl. CPC G01R 33/0047

(57) ABSTRACT

A device (e.g., a micromechanical resonator) can include an elongated housing having a first end, a second end, and a sidewall extending from the first end to the second end. A longitudinal opening can extend through the sidewall and extend from an axial center at the first end to the second end. The device can include a filament with a first end of the filament attached to the first end at the axial center. The device can include a first magnetic object with a second end of the filament attached to the first magnetic object. The device can include a second magnetic object and being spaced apart from the first magnet. The filament is configured to allow the first magnetic object to oscillate about a longitudinal axis of the elongated housing relative to the second magnetic object in response to a magnetic or electromagnetic field.

### CLAIM OF PRIORITY

This application claims priority under 35 USC § 119(e) to U.S. Patent Application Ser. No. 63/793,237, filed on Apr. 23, 2025, and U.S. Patent Application Ser. No. 63/793,271, filed on Apr. 23, 2025, the entire contents of which are hereby incorporated by reference.

### TECHNICAL FIELD

The present disclosure relates to passive micromechanical resonators that can produce a detectable signal in a magnetic or electromagnetic field that can be sensed externally of the micromechanical resonator to determine a position and/or orientation of micromechanical resonator.

### BACKGROUND

It is often desirable to track markers within an anatomy of a patient to diagnose medical conditions. Some techniques include imaging (e.g., an angiogram, ultrasound, tomography, etc.) to track the markers. Other techniques use electromagnetic sensors to track the markers. For example, an electromagnetic sensor can detect an electric or magnetic field generated by a marker and determine the position of the marker based on the detected electric or magnetic field.

### SUMMARY

Micromechanical resonators (MMR) also referred to as “MicroBot” devices are small magnetic resonator devices that can have a typical size of less than 10 mm in length and a diameter of 0.4-1.5 mm. However, sizes and diameters larger and smaller than these ranges can be used. Inside a housing, two or three magnetic objects (e.g., spherical permanent magnets, cylindrical permanent magnets, coils, etc.) are mounted in a way that one magnetic object can mechanically oscillate in a magnetic or electromagnetic field with a defined distance from the other magnetic object(s) due to an excitation of a resonance mode. The medical oscillation of the magnetic object can be detected externally of the micromechanical resonator by a sensor to determine the position and/or location of the of the micromechanical resonator. The micromechanical resonators receive energy from an external magnetic or electromagnetic field and do not need a wired connection to an energy supply. Micromechanical resonators are completely passive devices and do not require electronics.

The micromechanical resonators described herein include at least one free (e.g., non-fixed) magnetic object arranged in mechanical equilibrium with a second magnetic object (e.g., a second free magnetic object, a fixed magnetic object, etc.). In the presence of an external magnetic or electromagnetic field (e.g., generated by a transducer in a transmit mode), the mechanical equilibrium of the magnetic objects is disturbed, and the free magnetic object oscillates in a rotational mode relative to the second magnetic objects(s) at one or more resonant frequencies to produce a detectable signal (e.g., a pulse) within the magnetic or electromagnetic field. After turning off the magnetic or electromagnetic field, the free magnetic object continues oscillating in the rotational mode and continues emitting the detectable signal in the magnetic or electromagnetic field. The detectable signal can be sensed by a receiver (e.g., the transducer operating in a receive mode) and a computer system can determine one or more properties (e.g., a position, orientation, spatial (e.g., 2D or 3D) coordinates, etc.) of the micromechanical resonator based on detectable signals from the micromechanical resonator. Details of micromechanical resonators are described in U.S. Pat. Nos. 11,598,677 and 11,774,300, and U.S. Publication No. 2025/0127417A1 which are hereby incorporated by reference in their entirety. Additional micromechanical resonators are described in U.S. application Ser. No. 19/276,522 filed contemporaneously with this application which is also hereby incorporated by reference in its entirety.

Low friction between the oscillating magnetic object and objects (magnetic and non-magnetic) in contact with the oscillating magnetic object is important. Low friction helps to provide a long and measurable oscillation after turning off the external magnetic or electromagnetic field. For example, the oscillating magnetic object can be held in position by a thin filament which twists during oscillation of the oscillating magnetic object. Having a filament that is resilient (e.g., not brittle) in torsion (e.g., by a low torsional stiffness) and stiff under uniaxially loading can be helpful.

This disclosure describes a method for manufacturing a housing for the micromechanical resonator and fixtures (e.g., a carrier) for a fixed magnetic object inside the housing using 3D printing technologies. The housing includes a longitudinal opening (e.g., a slit) that allows insertion of a filament attached to a free magnetic object into the housing. The housing can also include a carrier for the fixed magnetic object. The carrier can be pre-aligned and positioned at a pre-determined distance inside the housing so that the fixed magnetic object remains a constant axial distance from the free magnetic object. The distance can be defined by an internal distance element. In some cases, the carrier is pre-aligned using a magnetic tool.

The dimensions and the precision for the fabrication are challenging and conventional manufacturing methods can be limiting. 3D printing with a 2-photon polymerization technology allows precise printing of features with a resolution down to 200 nm. 3D printing with a 2-photon polymerization technology in combination with a printing compatible design and geometry that allows for assembly of a two (or three) magnet resonator as described herein.

In a first aspect, a device includes an elongated housing having a first end, a second end, and a sidewall extending from the first end to the second end. A longitudinal opening extends through the sidewall of the elongated housing and extends from an axial center at the first end of the elongated housing to the second end of the elongated housing. The device includes a filament disposed in the elongated housing with a first end of the filament attached to the first end of the elongated housing at the axial center. A first magnetic object is disposed in the elongated housing with a second end of the filament attached to the first magnetic object. A second magnetic object is disposed in the elongated housing and being spaced apart from the first magnetic object. The filament is configured to allow the first magnetic object to oscillate about a longitudinal axis of the elongated housing relative to the second magnetic object in response to a magnetic or electromagnetic field (e.g., an external magnetic or electromagnetic field).

In some implementations, the longitudinal opening is configured to allow the filament to be inserted into the elongated housing with the second end of the filament attached to the first magnetic object prior to the first end of the filament being attached and fixed to the first end of the elongated housing at the axial center.

In some implementations, the longitudinal opening can be closed with the filament inside the elongated housing and the second end of the filament attached to the first magnetic object.

In some implementations, a magnetic axis of the first magnetic object is perpendicular to the longitudinal axis of the elongated housing, a magnetic axis of the second magnetic object is perpendicular to the longitudinal axis of the elongated housing, and the magnetic axis of the first magnetic object and the magnetic axis of the second magnetic object have opposite polarities.

In some implementations, the first magnetic object is configured to oscillate about the longitudinal axis of the elongated housing relative to the second magnetic object while maintaining a fixed axial distance to the second magnetic object in response to the magnetic or electromagnetic field.

In some implementations, the first magnetic object is configured to be rotated out of an equilibrium orientation in response to the magnetic or electromagnetic field to initiate the oscillation relative to the second magnetic object.

In some implementations, the first magnetic object is suspended in the elongated housing by the filament and does not contact the sidewall of the elongated housing.

In some implementations, the device includes a carrier disposed in the elongated housing, the second magnetic object being fixed in the carrier, and the carrier positioning the second magnet at a pre-determined distance from the first magnetic object.

In some implementations, the sidewall of the elongated housing includes a distance element configured to contact the carrier to position the second magnetic object at the pre-determined distance from the first magnetic object, the protrusion having a curved inner rim.

In some implementations, the second end of the filament is adhered to the first magnetic object by a cyanoacrylate, an epoxy-based adhesive, or a UV curable adhesive.

In some implementations, the first end of the filament is adhered to the first end of the elongated housing at the axial center by the cyanoacrylate, the epoxy-based adhesive, or the UV curable adhesive.

In some implementations, the device includes a second part, the second end of the filament being directly attached to the second part, and the second part being directly attached to the first magnetic object.

In some implementations, the second magnetic object is fixed relative to the elongated housing.

In some implementations, the filament is a first filament, and the device includes a second filament attached to the second magnetic object, the second filament being configured to allow the second magnetic object to oscillate about the longitudinal axis of the elongated housing relative to the first magnetic object in response to the magnetic or electromagnetic field.

In some implementations, the device includes a third magnetic object disposed between the first and second magnetic objects, the third magnetic object being fixed to the elongated housing.

In some implementation, the filament includes a Kevlar® fiber, an ultra-high density polyethylene fiber, a carbon fiber, or an aramid fiber.

In some implementations, a torsional stiffness of the filament is less than 1e-7 Nm/rad, and an axial stiffness of the filament is greater than 1 GPa.

In some implementations, the first magnetic object is a first permanent magnet, and the second magnetic object is a second permanent magnet.

In some implementations, the device does not require electronics to produce the oscillations. In a second aspect, a device includes an elongated housing including a longitudinally-extending opening. The device includes a filament having a first end fixed to the elongated housing within the longitudinally-extending opening. A first magnetic object is disposed in the elongated housing with a second end of the filament fixed to the first magnetic object. A second magnetic object is disposed in the elongated housing and being spaced apart from the first magnetic object. The filament is configured to allow the first magnetic object to oscillate about a longitudinal axis of the elongated housing relative to the second magnetic object.

In some implementations, the longitudinally-extending opening is configured to allow the filament to be inserted into the elongated housing with the second end of the filament attached to the first magnetic object prior to the first end of the filament being attached and fixed to the first end of the elongated housing.

In some implementations, the elongated housing includes a cylindrical portion having a diameter less than 2 mm and a length less than 4 mm.

In some implementations, the second end of the elongated housing defines a notch at the longitudinally-extending opening for guiding the filament into the longitudinally-extending opening.

In some implementations, a width of the longitudinally-extending opening is less than 15 micrometers larger than a diameter of the filament.

In some implementations, the filament includes a Kevlar® fiber, an ultra-high density polyethylene fiber, a carbon fiber, or an aramid fiber.

In some implementations, the first magnetic object is a first permanent magnet, and the second magnetic object is a second permanent magnet.

In some implementations, at least one of the first or second magnetic objects is a spherical permanent magnet.

In some implementations, the first and second permanent magnets are spherical, and the second spherical permanent magnet has a larger diameter than the first spherical permanent magnet.

In some implementations, at least one of the first or second magnetic objects is a cylindrical permanent magnet.

In some implementations, the second end of the filament is adhered to the first magnetic object by a cyanoacrylate, an epoxy-based adhesive, or a UV curable adhesive.

In some implementations, the second magnetic object is fixed to the housing.

In some implementations, the elongated housing is a substantially rigid monolithic structure.

In some implementations, at least one of the first or second magnetic objects includes a coil.

In a third aspect, a system includes one or more devices. Each device includes an elongated housing including a longitudinal opening extending from a first end of the elongated housing to a second end of the elongated housing. Each device includes a filament disposed in the elongated housing with a first end of the filament attached to the first end of the elongated housing. Each device includes a first magnetic object disposed in the elongated housing with a first end of the filament attached to the first magnetic object. Each device includes a second magnet object disposed in the elongated housing and spaced apart from the first magnetic object. The filament of each device is configured to allow the first magnetic object to oscillate relative to the second magnetic object at a resonant frequency in response to a magnetic or electromagnetic field (e.g., an external magnetic or electromagnetic field). Each device having a different resonant frequency.

In some implementations, each device has a different resonant frequency due to differing lengths of the filament between the first end of the elongated housing and the first magnetic object.

In some implementations, the system includes one or more electromagnetic sources external to the one or more devices, the one or more electromagnetic sources configured to provide respective magnetic or electromagnetic fields to excite the respective resonant frequencies.

In some implementations, the system includes one or more sensors external to the one or more devices, the one or more sensors being configured to determine respective positions of the one or more devices based on sensing the respective resonant frequencies.

In some implementations, the system includes a medical device. The one or more devices are disposed in the medical device, and the one or more sensors are configured to determine a position of the medical device based on sensing the respective resonant frequencies.

In some implementations, for each device, the longitudinal opening is configured to allow the filament to be inserted into the elongated housing with the second end of the filament attached to the first magnetic object prior to the first end of the filament being attached and fixed to the first end of the elongated housing.

In some implementations, for each device, a magnetic axis of the first magnetic object is perpendicular to a longitudinal axis of the elongated housing, a magnetic axis of the second magnetic object is perpendicular to the longitudinal axis of the elongated housing, and the magnetic axis of the first magnetic object and the magnetic axis of the second magnetic object have opposite polarities.

In a fourth aspect, a method includes attaching a first end of a filament to a first magnetic object. With the first end of the filament attached to the first magnetic object, the filament is inserted through a notched end of a longitudinal opening of an elongated housing to locate the first magnetic object and the filament within the elongated housing. After locating the first magnetic object and the filament within the elongated housing, a second magnetic object is inserted into the elongated housing at a position in which the second magnetic object is spaced apart from the first magnet.

In some implementations, a second end of the filament is attached to a first end of the elongated housing to fix a length of the filament and a resonant frequency in which the first magnetic object oscillates relative to the second magnetic object.

In some implementations, the method includes tuning the resonant frequency by varying the length of the filament between the first end of the elongated housing and the first magnetic object before attaching the second end of the filament to the first end of the elongated housing.

In some implementations, the elongated housing includes a cylindrical portion having a diameter less than 2 mm and a length less than 4 mm.

In some implementations, a width of the longitudinal opening is less than 15 micrometers larger than a diameter of the filament.

In some implementations, the longitudinal opening is only on one lateral side of the elongated housing.

In some implementations, the elongated housing has been 3D printed by 2-photon polymerization technology.

In some implementations, attaching the first end of the filament to the first magnetic object includes using an adhesive to adhere the first end of the filament to the first magnetic object.

In some implementations, the first end of the filament is adhered to the first magnetic object by a cyanoacrylate, an epoxy-based adhesive, or a UV curable adhesive.

In a fifth aspect, a method includes printing, using a 3D printing technology, an elongated housing having a longitudinal opening extending from a first longitudinal end of the elongated housing to a second longitudinal end of the elongated housing. The longitudinal opening for inserting a filament and a first magnetic object attached to the filament into the elongated housing. The method includes printing, using the 3D printing technology, a carrier configured to be disposed in the elongated housing, the carrier configured to hold a second magnetic object in a fixed position in the elongated housing and spaced apart from the first magnetic object.

In some implementations, the 3D printing technology is a 2-photon polymerization technology.

In some implementations, the elongated housing includes a distance element defined by a stepped portion of a sidewall of the elongated housing, the carrier configured to abut against the distance element when inserted into the elongated housing.

In some implementations, the elongated housing includes a notch element at the second longitudinal end for guiding the filament into the longitudinal opening.

While specific implementations have been described for specific aspects, any of the above-noted implementations and features of the above aspects can be combined with any of the other aspects. For example, features and/or implementations described with reference to the first aspect can be used with second aspect, and vice versa, etc. In addition, any combination of the above-noted aspects is also within the scope of this disclosure.

The technology described herein can include one or more of the following advantages.

The micromechanical resonators described herein do not require electronics and are completely passive devices. This feature allows them to be used in remote applications. This feature, coupled with the fact that micromechanical resonators described herein can be very small (on the order of millimeters), allows the micromechanical resonators to be used in applications where other sensors cannot (e.g., underneath human skin, within an anatomy of a human, etc.).

The longitudinal opening in the housing improves the manufacturability of the miniature micromechanical resonator. Without a longitudinal opening, one would need to suction the filament through a hole in the housing which can be difficult. The longitudinal opening, especially with the notched end, is helpful for inserting the filament with the free magnetic object attached thereto into the housing.

Printing the housing using a 2-photon polymerization technology can allow the housing to be printed with the longitudinal opening directly instead of requiring a secondary manufacturing method to produce the longitudinal opening. The longitudinal opening may be on the order of microns wide, which can be difficult to manufacture otherwise.

Using a carrier within the housing allows a fixed magnetic object to be pre-aligned in the housing (e.g., to concentrically position the fixed magnetic object within the housing and to position the fixed magnetic object a pre-determined distance from the free magnetic object). This is helpful during assembly to increase the likelihood of proper alignment and consistency between different micromechanical resonators.

Using a filament with a low torsional stiffness while being resilient to rotation and bending can be helpful to limit the filament's influence on the resonant frequency of the free magnetic device and allow flexibility in the assembly process. For example, if too stiff of a filament is used, the filament can break during assembly and would need to be replaced.

Designing the housing with radial clearance between the free magnetic object and the sidewall of the housing is helpful to limit the likelihood of the free magnetic object hitting the sidewall of the housing while oscillating in the rotational resonance mode. While it has been found that gravitational influence on the free magnetic object reduces with the size of the micromechanical resonators, it is nevertheless helpful to provide clearance between the free magnetic object and the sidewall of the housing. For example, a free magnetic object in a large micromechanical resonator (e.g., on the order of 100 cm) may impact the sidewall of the housing during shaking of the micromechanical resonator. However, a free magnetic object in a small micromechanical resonator (e.g., on the order of 1 mm) is very unlikely to impact the sidewall of the housing during shaking of the micromechanical resonator due to the relative strength of the magnetic force compared to the gravitational force.

The details of one or more implementations are set forth in the accompanying drawings and the description herein. Other features, objects, and advantages will be apparent from the description and drawings, and from the claims.

### DESCRIPTION OF THE DRAWINGS

FIG. 1 shows a micromechanical resonator being used in a medical environment.

FIG. 2 is a schematic of a micromechanical resonator with a free magnetic object and a fixed magnetic object.

FIG. 3 is a time-domain plot illustrating voltages of an external magnetic or electromagnetic field.

FIG. 4 is a frequency-domain plot illustrating resonances of a micromechanical resonator.

FIGS. 5 and 6 are views of a housing of a micromechanical resonator.

FIG. 7 is a view of a curved rim of a housing of a micromechanical resonator.

FIG. 8 shows a housing having a V-shape notch.

FIGS. 9 and 10 are views of a carrier of a micromechanical resonator.

FIGS. 11, 12, and 13 are views of a micromechanical resonator.

FIG. 14 is an example method for assembling a micromechanical resonator.

FIGS. 15 and 16 are views of a carrier of a micromechanical resonator.

FIG. 17 is an example method for manufacturing a housing and a carrier of a micromechanical resonator.

FIG. 18 is a schematic of a micromechanical resonator with cylindrical permanent magnets.

FIG. 19 is a schematic of a micromechanical resonator with three magnetic objects.

FIG. 20 is a schematic of a micromechanical resonator with an attachment device.

FIG. 21 is a schematic of a micromechanical resonator with two movable magnetic objects.

FIG. 22 shows an example computer device and example mobile computer device which can be used to implement the techniques described herein.

Like reference numbers represent corresponding parts throughout.

### DETAILED DESCRIPTION

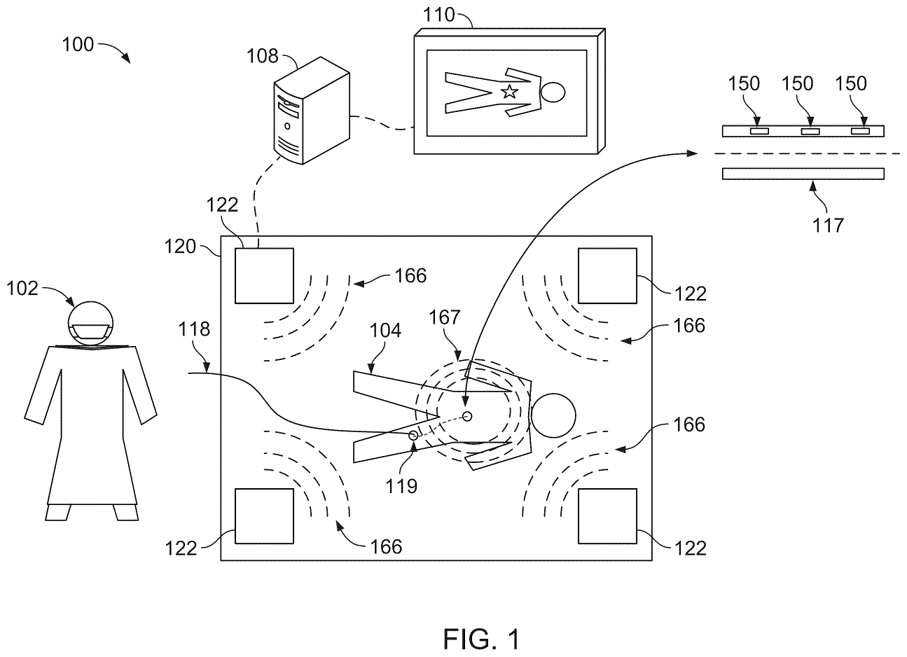

FIG. 1 shows a medical environment 100 where a medical professional 102 desires to know the position and/or orientation of a device 150 (e.g., a micromechanical resonator, marker device, sensor, etc.) within the anatomy of a patient 104 during a medical procedure (e.g., a minimally invasive procedure). For example, in the example represented in FIG. 1, the medical professional 102 has inserted a catheter 118 into the patient 104 via an insertion site 119. In this example, three micromechanical resonators 150 are embedded in a sidewall 117 of the catheter 118. In other examples, a single micromechanical resonator 150 can be used, or more generally one or more micromechanical resonators 150 can be used.

Encapsulating the micromechanical resonator 150 in the sidewall 117 of the catheter 118 can protect the magnets and inner components of the micromechanical resonator 150 from being exposed to bodily fluids and/or otherwise being contaminated. The catheter 118 is generally a thin-walled flexible tube formed of non-magnetic bio-compatible material (polyethylene, silicone). The micromechanical resonator 150 can be very small (e.g., 1 mm in wall thickness or less) so it can fit inside the catheter 118 and allow the functionality of the catheter 118. In some examples the micromechanical resonator 150 is embedded in the sidewall 117 of the catheter 118. In other applications, the micromechanical resonator 150 is disposed on an interior of the sidewall 117 of the catheter 118 or disposed on an exterior of the sidewall 117 of the catheter 118. In other applications, the micromechanical resonator 150 can be orally consumed by the patent while being able to withstand and survive passing through the digestive tract of the patient. In some examples, the micromechanical resonator 150 can be embedded into the patient (e.g., via surgery).

In the example shown in FIG. 1, the patient 104 is laying on a bed 120 with a plurality of electromagnetic transducers 122 embedded in the bed 120. Each of the transducers 122 are connected to a computer system 108 and generally include one or more coils to produce and transmit an electro-magnetic field 166. Each transducer 122 is arranged to produce the electromagnetic field 166 in the vicinity of the patient 104. The transducers 122 can be configured to operate in both a transmit mode to transmit the electromagnetic field 166 and in a receive mode to receive an electro-magnetic field 167.

In some implementations, each transducer 122 produces a different electromagnetic field 166 so that distances and orientations between the micromechanical resonator 150 and the respective transducers 122 can be determined by the computer system 108. Each transducer 122 can also provide a different magnetic field. In some examples, the computer system 108 controls the transducers 122 by controlling them on and off so that only one of the transducers 122 is transmitting an electromagnetic field 166 at a given time. While the transducers 122 are embedded in the bed 120 in this example, in some implementations, the transducers 122 are embedded elsewhere (e.g., in a C-arm machine).

When the transducers 122 are in receive mode, the transducers 122 sense a magnetic or electromagnetic signal 167 produced by the micromechanical resonator 150. The received (or sensed) magnetic or electromagnetic signal 167 is then processed by the computer system 108 to determine a position and/or orientation of the micromechanical resonator 150 within the anatomy of the patient 104.

In some cases, the computer system 108 controls a display 110 to present a representation of the determined position, orientation, etc. of the micromechanical resonator 150 within the anatomy of the patient 104. The computer system 108 includes one or more processors to determine spatial characteristics (e.g., the position and/or orientation) of the micromechanical resonator 150 within the anatomy of the patient 104 and control the display 110. The computer system 108 and/or medical professional 102 uses the spatial characteristics of the micromechanical resonator 150 within the anatomy of the patient 104 to determine the position and/or orientation of the catheter 118. This information can assist the medical professional 102 in navigating the catheter 118 through a tortuous anatomy of the patent 104 and determining when the catheter 118 has arrived at its destination within the patent 104. After use, the catheter 118 including the micromechanical resonators 150 can be disposed.

Preferably, the micromechanical resonator 150 is relatively small (e.g., typically on the order of millimeters (mm)). These dimensions allow the micromechanical resonator 150 to be embedded in medical instruments (e.g., the catheter 118) to assist in determining the position and/or orientation of the medical instrument. Further, the small nature of the micromechanical resonator 150 allows the catheter 118 to maintain a relatively small bending radius to assist in navigating the catheter 118 through the tortuous anatomy of the patent 104. As such, it can be preferable to taper one or more ends of the micromechanical resonator 150 to avoid limiting a bending radius of the medical instrument, for example. The micromechanical resonator 150 can also be used in a marker for tracking (e.g., to determine the position and/or orientation of the marker over time within the anatomy of the patient 104).

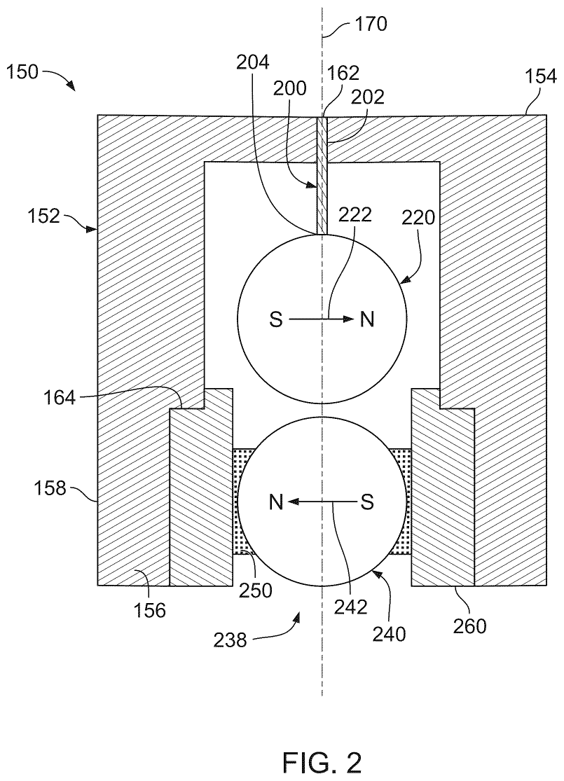

FIG. 2 shows an example micromechanical resonator 150 with two magnetic objects implemented as spherical permanent magnets. The micromechanical resonator 150 includes a housing 152 (e.g., an elongated housing, or casing) having a first end 154, a second end 156, and a sidewall 158 extending from the first end 154 to the second end 156. The housing 152 can be non-magnetic and substantially rigid (e.g., formed of plastic, 3D printed plastic, etc.). The housing 152 can be cylindrical having a diameter less than 2 mm (e.g., between 0.4 and 1.5 mm) and a length less than 2 mm (e.g., between 1 and 3 mm). The housing 152 can be 3D printed by a 2-photon polymerization technology. One or more ends of the housing 152 can be tapered. The housing 152 can be monolithic. The housing 152 can be a substantially rigid monolithic structure.

The micromechanical resonator 150 includes a torsionally resilient slender member 200 (e.g., a filament, a fiber, a thread, a thread-like structure, a plurality of fibers, etc.) disposed in the housing 152 with a first end 202 of the filament 200 attached or fixed to the first end 154 of the housing 152 at the axial center 162. The torsionally resilient slender member 200 can be cylindrical. The torsionally resilient slender member 200 will be referred to as a filament 200 throughout this disclosure but it should be understood that the torsionally resilient slender member 200 need not be limited to a filament 200.

The filament 200 can be or include a Kevlar® fiber, an ultra-high density polyethylene fiber, or a carbon fiber (e.g., a carbon nanofiber). The filament 200 can be or include one or more organic fibers in the aromatic polyamide family (aramids). For example, the filament 200 can include an aromatic polyamide (e.g., aramid) fiber. The filament 200 can be or include an aramid fiber (e.g., a Kevlar® aramid fiber).

Preferably, a torsional stiffness of the filament 200 is as small as possible (e.g., less than 1e-7 Nm/rad). A low torsional stiffness can limit the filament's 200 influence on torsional vibration modes. The torsional stiffness of the filament 200 depends on the size of the magnetic object that performs the rotational oscillation (e.g., the first magnetic object 220). It can be preferred to have the torsional stiffness of the filament 200 less than 1/10 of the stiffness due to the magnetic field. For example, for magnetic objects implemented as 0.5 mm diameter spheres the stiffness due to the magnetic field is about 1e-6 Nm/rad. This stiffness scales with the volume of the oscillating magnetic object. The torsional stiffness of the filament 200 can be reduced by using as thin of a filament 200 as possible.

Preferably, the axial stiffness of the filament 200 is as large as possible (e.g., greater than 1 GPa). Having a large axial stiffness can increase the likelihood of maintaining a constant spacing between two magnetic objects and the limit the likelihood of uniaxial vibration modes being excited during operation. In some cases, using materials with an axial stiffness between 10 GPa and 20 GPa is be preferred.

Preferably, the tensile strength of the filament 200 is as large as possible (e.g., greater than 1 GPa). Having a large tensile strength can decrease the likelihood of breakage of the filament 200 when the magnetic objects are forced towards one another. In some cases, materials having a tensile strength exceeding 1 GPa (e.g., exceeding 2 GPa) is be preferred. Since the torsionally resilient slender member 200 is thin, a high tensile strength is generally needed.

The bending stiffness of the filament 200 is typically not a design constraint. The inventors have found success in using materials with wide ranges of bending stiffnesses. The first end 154 of the filament 200 can be adhered (e.g., glued) to the housing 152 (e.g., by a cyanoacrylate, an epoxy-based adhesive, or a UV curable adhesive). The axial length of the filament 200 within the housing 152 is fixed. An axial length of the filament 200 inside the housing 152 can be between 300 and 800 μm. For example, the axial length of the filament 200 inside the housing 152 can be between 300 and 500 μm. A diameter of the filament 200 can be between 4 and 20 μm. For example, the diameter of the filament 200 can be between 4 and 12 μm.

The micromechanical resonator 150 includes a first magnetic object 220 (e.g., a first permanent magnet or coil) disposed in the housing 152 with a second end 204 of the filament 200 connected (e.g., directly or indirectly attached or fixed) to the first magnetic object 220. The second end 204 of the filament 200 can be adhered (e.g., glued) to the first magnetic object 220 (e.g., by a cyanoacrylate, an epoxy-based adhesive, or a UV curable adhesive). A magnetic axis 222 of the first magnetic object 220 is perpendicular to a longitudinal axis 170 of the housing 152, with a north and south pole oriented perpendicular to the longitudinal axis 170. The first magnetic object 220 has a magnetic dipole. The first magnetic object 220 can be a permanent magnet. The first magnetic object 220 can be a spherical permanent magnet with the second end 204 of the filament 200 attached or fixed to an equator of the first spherical permanent magnet 220, the equator extending around the magnetic axis 222 of the first spherical permanent magnet 220.

The micromechanical resonator 150 includes a restoring torque unit 238 disposed in the housing 152 and being spaced apart from the first magnetic object 220. The restoring torque unit 238 can be a second magnetic object 240 (e.g., a second permanent magnet, coil, etc.). The second magnetic object 240 can be fixed relative to the housing 152, e.g., by an adhesive 250. The adhesive 250 can be a cyanoacrylate, an epoxy-based adhesive, or a UV curable adhesive. A magnetic axis 242 of the second magnetic object 240 is perpendicular to the longitudinal axis 170 of the housing 152, with a north and south pole oriented perpendicular to the longitudinal axis 170. The second magnetic object 240 has a magnetic dipole. The magnetic axis 222 of the first magnetic object 220 and the magnetic axis 242 of the second magnetic object 240 have opposite polarities (e.g., the respective north and south poles are oriented in opposite directions to one another). In this way, the magnetic axis 242 of the second magnetic object 240 is anti-parallel to the magnetic axis 222 of the first magnetic object 220. The second magnetic object 240 can be a spherical or cylindrical permanent magnet having a larger diameter than the first magnetic object 220.

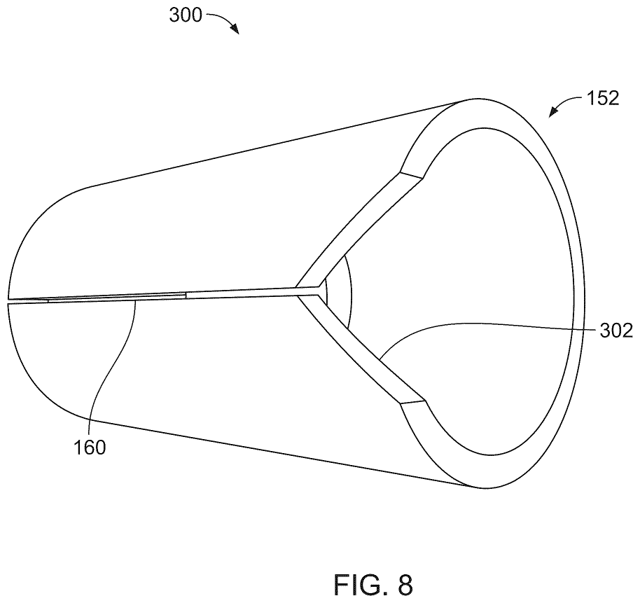

Depending on the size and geometry of the second magnetic object 240, a first part 260 (e.g., a retainer or carrier) can be used to support the second magnetic object 240 and position the second magnetic object 240 at a fixed axial distance from the first magnetic object 220. The carrier 260 can be inserted into the housing 152 from the second end 156 and contact a distance element 164 (e.g., a protrusion or wall) of the housing 152 with the second magnetic object 240 disposed within the carrier 260 to increase the likelihood of consistent positioning of the second magnetic object 240 in each housing 152. With the carrier 260 in contact with the distance element 164 of the housing 152, an adhesive (e.g., a cyanoacrylate, an epoxy-based adhesive, or a UV curable adhesive) can be applied between the carrier 260 and the housing 152 to fix the carrier 260 and thus the second magnetic object 240 relative to the elongate housing 152. The second magnetic object 240 can be adhered to the carrier 260 to fix the second magnetic object 240 to the carrier 260 using a cyanoacrylate, an epoxy-based adhesive, or a UV curable adhesive. The carrier 260 fixes the second magnetic object 240 at a pre-determined axial distance from the first magnetic object 220 and centers the second magnetic object 240 radially within the housing 152. The distance element 164 can be a stepped portion of the sidewall 158 of the housing 152. The distance element 164 can be defined by an additional part that can be removably attached to a groove on the inner surface of the housing 152 (e.g., a snap ring). The distance element 164 can have a curved internal rim 165 (see, e.g., FIGS. 6 and 7). A curved internal rim 354 can be helpful to guide the filament 200 into position within the housing 152, as described below.

In equilibrium, the first and second magnetic objects 220, 240 align with anti-parallel orientation of their magnetization. An external magnetic or electromagnetic field pulse can be used to start a mechanical resonance via a rotational oscillation of the first magnetic object 220 relative to the second magnetic object 240. The filament 200 resiliently deforms (e.g., twists) by torsion to allow the first magnetic object 220 to oscillate relative to the second magnetic object 240 in response to the external magnetic or electromagnetic field pulse.

The first magnetic object 220 can rotate out of the equilibrium orientation in response to the external magnetic or electromagnetic field pulse to initiate rotational oscillations relative to the second magnetic object 240. The first magnetic object 220 is configured to rotate (e.g., oscillate) about the longitudinal axis 170 of the housing 152 relative to the second magnetic object 240 while maintaining a fixed distance to the second magnetic object 240 in response to the external magnetic or electromagnetic field pulse. The first magnetic object 220 is suspended in the housing 152 by the filament 200 and preferably does not contact the sidewall 158 of the housing 152 during oscillations relative to the second magnetic object 240 in response to the external magnetic field pulse. Preferably, the first magnetic object 220 oscillates amount an angle greater than 20 degrees relative to the second magnetic object 240. Importantly, the micromechanical resonator 150 does not require electronics to produce the oscillations and is completely passive.

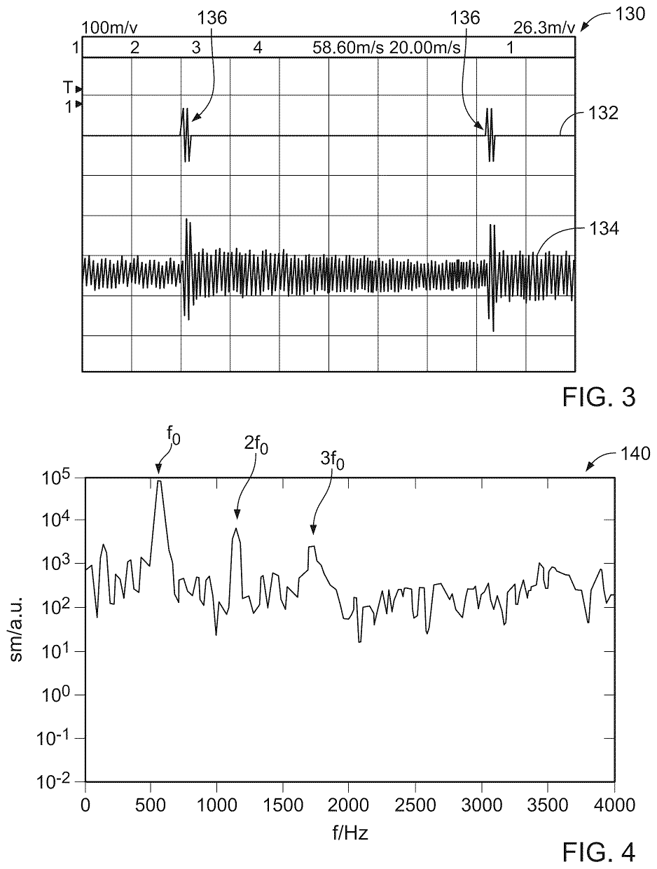

FIG. 3 is a time-domain plot 130 illustrating voltages of the external magnetic or electromagnetic field 166. The one or more processors of the computer system 108 generate transmit pulses (e.g., upper signal 132 in FIG. 3) that are amplified using an audio amplifier and then sent to transmit coils of the transducers 122. FIG. 3 shows, besides the transmit pulses 132, the induced voltage 134 in a receive coil of the transducers 122. The spacing of the excitation pulses 136 can be continuously adjusted by the one or more processors of the computer system 108. Once the transducers 122 measure the time-domain induced voltage 134 representing the magnetic field 167, the computer system 108 receives the induced voltage 134 and performs a Fast Fourier Transform (FFT) on the time-domain signal induced voltage 134 to produce a frequency-domain signal representing the magnetic field 167.

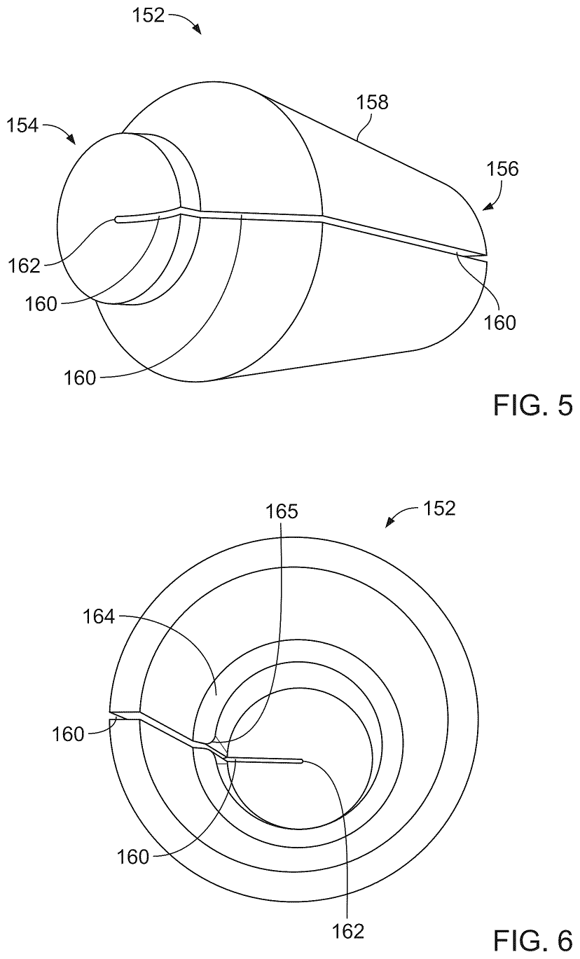

FIG. 4 is a frequency-domain plot 140 illustrating example detected resonances of the micromechanical resonators. The one or more resonance frequencies of the oscillating magnet are detectable by the computer system 108. Specifically, the fundamental frequency of oscillation of the first magnetic object 220 relative to the second magnetic object 240 is represented as f₀, with second and higher harmonics represented as 2f₀, 3f₀, etc. In some implementations, the computer system 108 determines the magnitude of the peaks in the time-domain or frequency-domain and uses these peaks to determine the position and/or orientation of the micromechanical resonator 150.

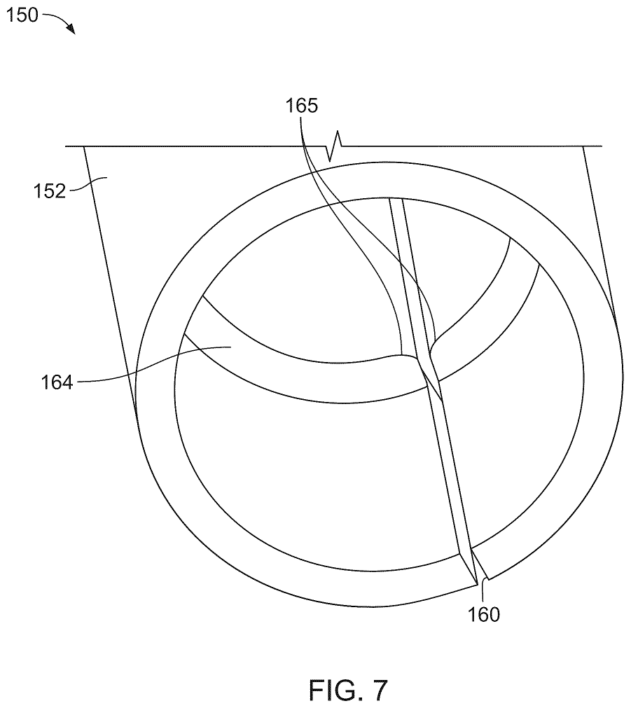

FIGS. 5, 6, and 7 show perspective views of the elongate housing 152. A longitudinal opening 160 (or longitudinally-extending opening) extends through the sidewall 158 of the housing 152 from the axial center 162 at the first end 154 of the housing 152 to the second end 156 of the housing 152. The longitudinal opening 160 is configured to allow the filament 200 to be inserted into the housing 152 with the second end 204 of the filament 200 attached to the first magnetic object 220 prior to the first end 202 of the filament 200 being attached and fixed to the first end 154 of the housing 152 at the axial center 162. The longitudinal opening 160 can be closed after assembly (e.g., by covering with an adhesive, or by encapsulating the entire housing 152 in a wrap or another part).

The first end 154 of the filament 200 is fixed to the housing 152 within the longitudinal opening 160. In some examples, a width of the longitudinal opening 160 at the axial center is larger than a remainer of the longitudinal opening 160 to assist during assembly of the device 150 by ensuring the filament 200 has arrived at the axial center 162 and by providing clearance for an adhesive to secure the filament 200 to the elongate housing 152 at the axial center 162.

A circular hole can be located at the axial center 162 within the longitudinal opening 160, the circular hole having a diameter larger than a width of the remainder of the longitudinal opening 160 (e.g., the circular hole can be enlarged relative to the remainder of the longitudinal opening 160). The longitudinal opening 160 can extend longitudinally to the axial center 162 where the hole is located. The hole can guide the filament 200 into position at the axial center 162 of the first end 154.

In some examples, the longitudinal opening 160 is on the order of microns wide (e.g., between 1-100 microns wide). In some examples, a circumferential gap defined by the longitudinal opening 160 is between 1 and 50 microns wide. In some examples, the circumferential gap defined by the longitudinal opening 160 is on the order of nanometers (e.g., between 100 nanometers and 1 micron thick). A width in the remainder of the longitudinal opening 160 (e.g., as measured in a circumferential direction relative to the longitudinal axis 170) can be less than 20 micrometers larger than a diameter of the filament 200. In some cases, the width in the remainder of the longitudinal opening 160 is less than 15 micrometers larger than the diameter of the filament 200. In some cases, the width in the remainder of the longitudinal opening 160 is less than 10 micrometers larger than the diameter of the filament 200.

The longitudinal opening 160 can be located on only one lateral side of the housing 152. The longitudinal opening 160 can be a longitudinal slit 160. The longitudinal opening 160 can be formed by 3D printing such that the elongate housing is printed to include the longitudinal opening 160 directly using a 2-photon polymerization technology. For example, a secondary manufacturing operation (e.g., wire electrical discharge machining (EDM), etc.) is not required). The housing 152 is open to ambient conditions due to the longitudinal opening 160. Embedding the housing 152 in a medical device can be used to seal the housing 152. As noted above, the longitudinal opening 160 can be closed after assembly with the filament 200 inside the housing 152 and the second end 204 of the filament 200 attached to the first magnetic object 220. In some examples, operating the micromechanical resonator 150 with air inside the housing 152 can be preferable to vacuum conditions with the housing 152 due to the electrostatic charge of the magnet objects. With no air there is no humidity, which can limit the flow electrostatic charge.

The housing 152 can include a distance element 164 (e.g., a protrusion or a wall) located on an interior of the housing 152. The distance element 164 is used to position a carrier in a fixed position within the housing 152, as described below. The distance element 164 can have a curved internal rim 165 for assisting in guiding the filament 200 into position. The curved internal rim 165 is curved and may not have any sharp edges. In some cases, sharp edges can damage or break a filament so it can be preferable to avoid sharp edges for parts in contact with the filament during assembly. When using the curved internal rim 165, the filament 200 can be inserted around the curved internal rim 165 and into the longitudinal opening 160 when feeding the filament 200 with the first magnetic object 220 attached thereto into the housing 152 and the filament 200 along the longitudinal opening 160.

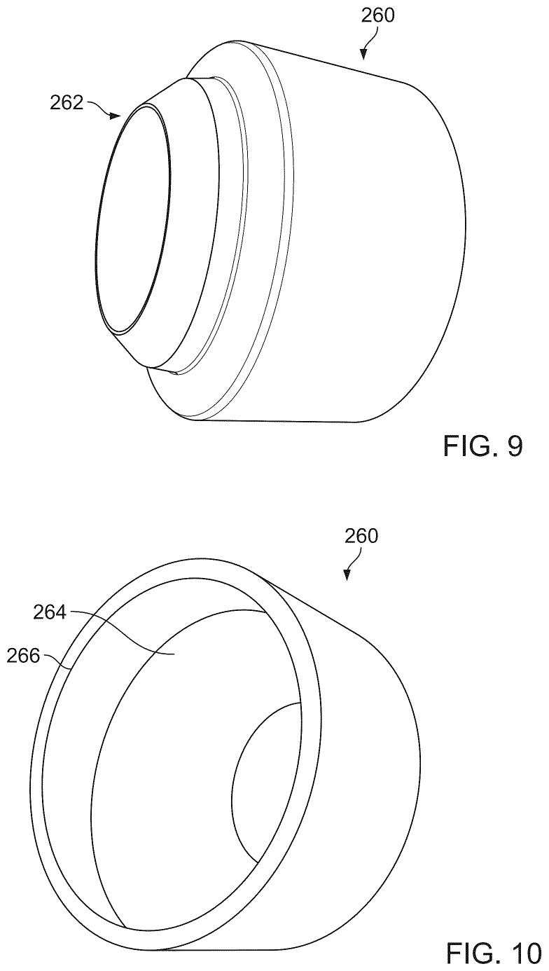

FIG. 8 shows an elongated housing 300 for a micromechanical resonator 150. The housing 300 is substantially the same as the housing 152 except that the elongated housing 300 includes a notch 302 at the second end 156. The second end 156 of the housing 300 defines the notch 302 at the longitudinal opening 160 for guiding the filament 200 into the longitudinal opening 160. The notch 302 can be tapered and/or V-shaped. The notch 302 can have two angled portions that allow the filament 200 to be guided to the longitudinal opening 160 during installation. It can be difficult to insert the filament 200 into small longitudinal openings that are on the order nanometers or microns wide without a notch to guide the filament 200.

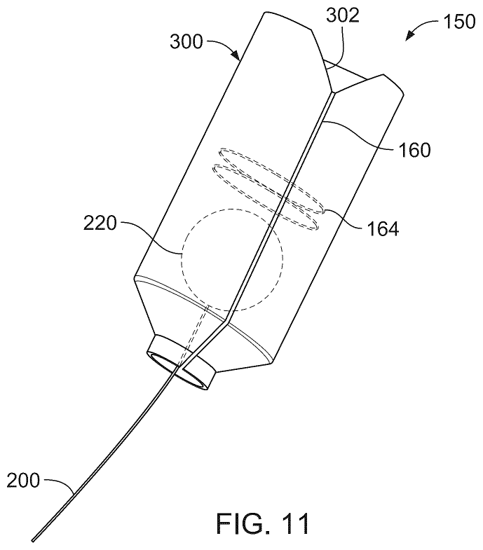

FIGS. 9 and 10 show an example of a first part 260 (or carrier) that can hold the second magnetic object 240 in a fixed position with the housing 152. The carrier 260 includes a first end 262 that contacts the distance element 164 inside the housing 152 (or the housing 300) for positioning the second magnetic object 240 within the housing 152 during assembly. The carrier 260 defines a receptacle 264 for the second magnetic object 240. When the second magnetic object 240 is a spherical permanent magnet, the carrier 260 can have a wall height measured along the longitudinal axis 170 that is slightly larger than a half diameter of the spherical permanent magnet 240. When the carrier 260 is positioned in the housing 152 with the first end 262 contacting the distance element 164 of the housing 152, a second end 266 of the carrier 260 can be substantially flush with the second end 156 of the housing 152. An adhesive (e.g., a cyanoacrylate, an epoxy-based adhesive, or a UV curable adhesive) can be used to adhere the carrier 260 into position within the housing 152 before or after the second magnetic object 240 is inserted into the carrier 260. The carrier 260 can be monolithic. The carrier 260 can formed of a plastic material and be substantially rigid. The carrier 260 can be substantially rigid monolithic structure.

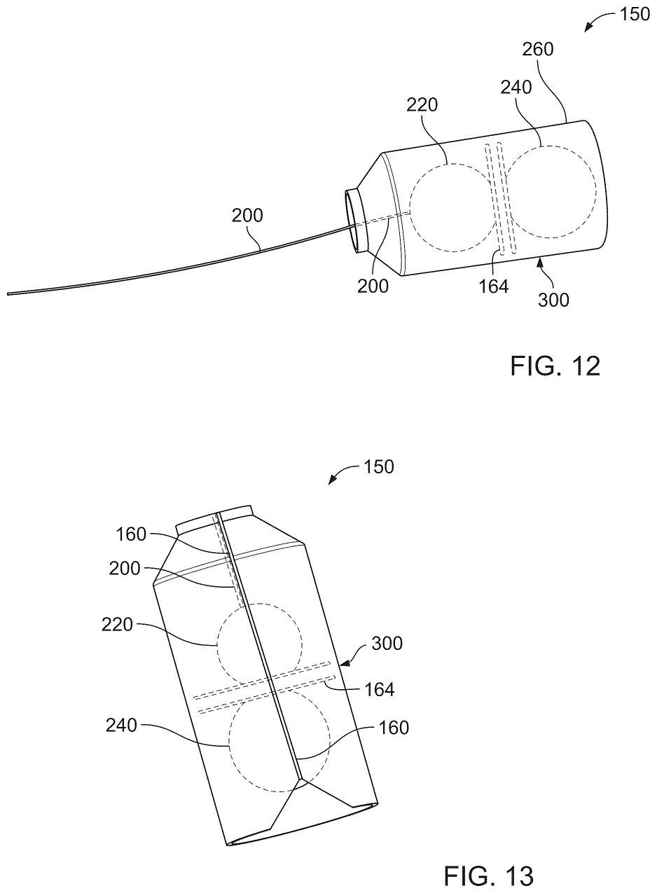

FIGS. 11-13 are views of an example implementation of the micromechanical resonator 150. The micromechanical resonator 150 shown in FIGS. 11-13 is substantially similar to the micromechanical resonator 150 shown in FIGS. 5-10 and therefore the Arabic numerals used with reference to FIGS. 5-10 are being reused for FIGS. 11-13. FIGS. 11-13 will be referenced along with an example method for assembling the micromechanical resonator 150.

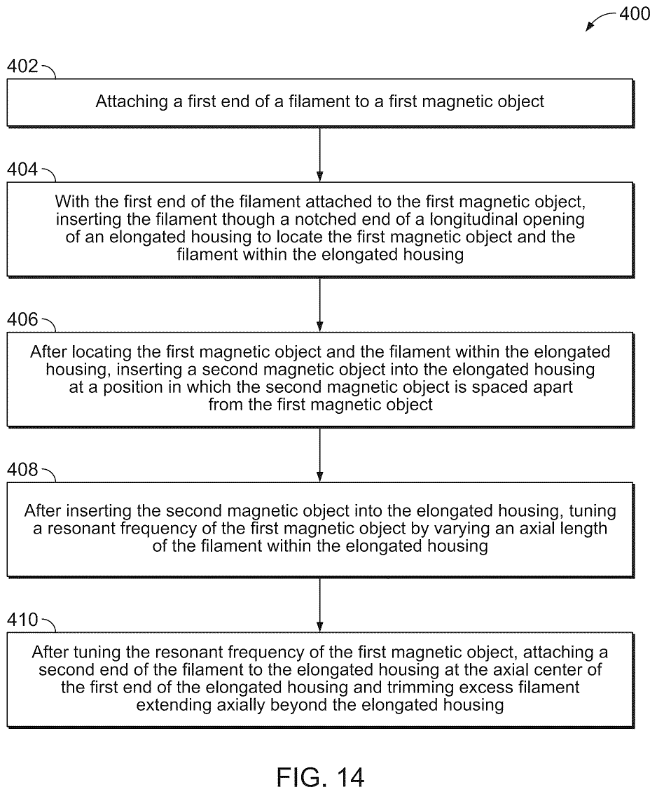

FIG. 14 is an example method 400 for assembling the micromechanical resonator 150. At step 402, the method 400 includes attaching a first end of a filament to a first magnetic object. For example, referring to FIG. 2, the end 204 of the filament 200 is attached to the first magnetic object 220. In some cases, an adhesive is used to glue the first end of the filament 200 to the first magnetic object 220. In some cases, the adhesive is a cyanoacrylate, an epoxy-based adhesive, or a UV curable adhesive.

At step 404, the method 400 includes with the first end of the filament attached to the first magnetic object, inserting the filament though a notched end of a longitudinal opening of an elongated housing to locate the first magnetic object and the filament within the elongated housing. For example, as shown in FIG. 11, the filament 200 has been inserted through the notched end 302 of the housing 300 to locate the first magnetic object 220 and the filament 200 within the housing 300. While a portion of the filament 200 extends beyond the housing 300 in FIG. 11, this excess portion is trimmed off during assembly, preferably after resonant frequency tuning (e.g., after step 408 as described below). In some cases, the filament 200 is positioned within a hole at the axial center 162 of the housing 300.

At step 406, the method 400 includes after locating the first magnetic object and the filament within the elongated housing, inserting a second magnetic object into the elongated housing at a position in which the second magnetic object is spaced apart from the first magnetic object. For example, as shown in FIGS. 12 and 13, the second magnetic object 240 has been inserted into the housing 300. In some cases, the second magnetic object 240 is fixed in position relative to the housing 300 (e.g., by an adhesive as described above). In some examples, the second magnetic object 240 is inserted into a carrier 260, and the carrier 260 is inserted into the housing 300, as described above.

At step 408, the method 400 includes after inserting the second magnetic object into the elongated housing, tuning a resonant frequency of the first magnetic object by varying an axial length of the filament within the elongated housing. For example, a resonant frequency in which the first magnetic object 220 oscillates relative to the second magnetic object 240 can be tuned by varying an axial length of the filament 200 within the housing 300 in response to excitation frequencies. For example, the approach described with reference to FIGS. 3 and 4 can be used to measure the resonant frequency of the of the first magnet. The resonant frequency will depend on the size of the first and second magnetic objects 220, 240, the spacing between them, and the mechanical properties of the filament 200. Controlling the length of the filament 200 within the housing 300 (e.g., the axial spacing between the first end 154 of the housing 300 and the first magnetic object 220) can directly affect the resonance frequency in which the first magnetic object 220 oscillates in response to an external magnetic or electromagnetic field.

At step 408, the method 400 includes after tuning the resonant frequency of the first magnetic object, attaching a second end of the filament to the elongated housing at the axial center of the first end of the elongated housing and trimming excess filament extending axially beyond the elongated housing. For example, as shown in FIG. 13, the excess filament 200 has been trimmed from the housing 300 and the filament 200 has been glued to the housing 300 so that the axial spacing between the first magnetic object 220 and the second magnetic object 240 remains constant (e.g., becomes fixed). The resonant frequency of the first magnetic object 220 remains fixed after the filament 200 has been glued to the housing 300. In addition, the axial spacing between the first magnetic object 220 and the second magnetic object 240 remains fixed after the filament 200 has been glued to the housing 300.

In some examples, the first magnetic object 220 is a small spherical permanent magnet and the second magnetic object 240 is a larger spherical permanent magnet. In some examples, the second magnetic object 240 is a spherical permanent magnet having a larger diameter than the a spherical permanent magnet of the first magnetic object 220. FIG. 12 shows both spherical magnets 220, 240 inside the housing 300. In some examples, the housings 152 and 300 and the carrier 260 are translucent. In some examples, the carrier 260 is a hollow cylinder.

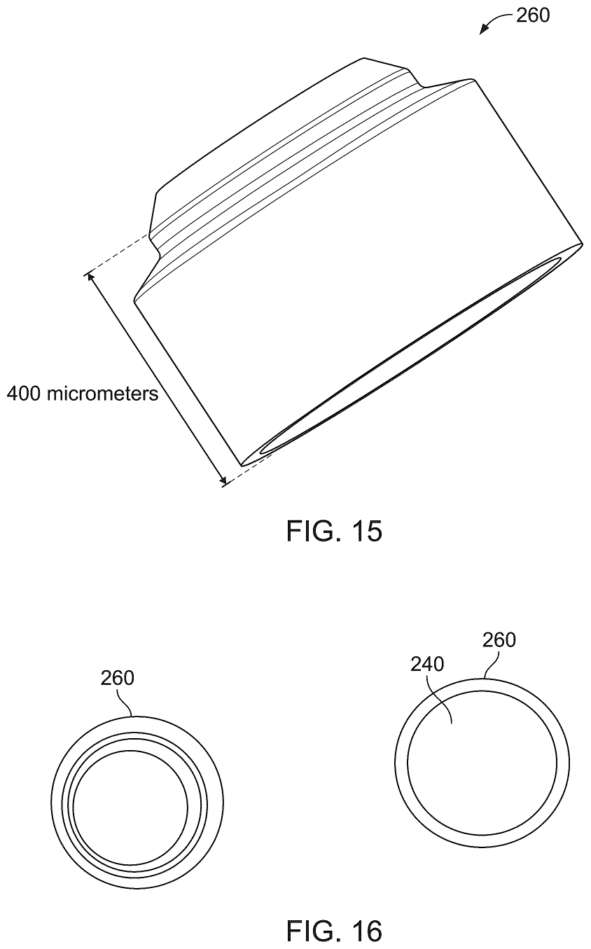

FIG. 15 shows that the height of the carrier 260 is about 400 μm. In some examples, the height of the carrier 260 can be slightly larger (10-50% larger) than a half sphere radius of a spherical permanent magnet of the second magnetic object 240 to be inserted into the carrier 260.

FIG. 16 shows the carrier 260 before (on the left) and after (on the right) the second magnetic object 240 is positioned in a receptacle of the carrier 260. In some examples, disposing the second magnetic object 240 in the carrier 260 pre-aligns the second magnetic object 240 in the carrier 260. In some examples, pre-aligning the first magnetic object 220 and/or second magnetic object 240 in the carrier 260 can be performed using a magnetic tool.

For example, a magnetic tool with a large magnet (e.g. below the carrier 260) can help to ensure the correct magnetic orientation of the second magnetic object 240 during assembly. For example, disposing the second magnetic object 240 on a large magnet with a known orientation allows the large magnet to magnetically align the magnetic axis of the second magnetic object 240 during assembly. In some examples, the magnetic tool can be used with the first magnetic object 220 as well. This can be helpful, for example, during filament attachment to the first magnetic object 220 so an assembler knows where to attach the filament 200 to the first magnetic object 220 to ensure the filament 200 is correctly located on the first magnetic object 220 (e.g., at an equator of the first magnetic object 220, etc.)

Once assembled, the first magnetic object 220 is disposed in the housing 152 with a first end 202 of the filament 200 adhered (e.g., glued) to the first magnetic object 220 and a second end 204 of the filament 200 adhered to the hole at the axial center 162 of the first end 154 of the housing 152. The first magnetic object 220 is movable relative to the housing 152. The second magnetic object 240 is disposed in the carrier 260. The carrier 260 is positioned at a pre-determined distance inside the housing 152. The distance is defined by an internal distance element 164 (e.g., the protrusion or the wall of the housing 152). In some embodiments, the second magnetic object 240 is fixed relative to the housing 152.

In some embodiments, the first magnetic object 220 can oscillate (e.g., rotate about the axis of the filament 200 and/or the longitudinal axis 170 of the housing 152) in a magnetic or electromagnetic field with a pre-determined axial distance to the second magnetic object 240 while the second magnetic object 240 remains fixed relative to the housing 152. The oscillation of the first magnetic object 220 can be detected by a sensor and used to determine a position of the micromechanical resonator 150.

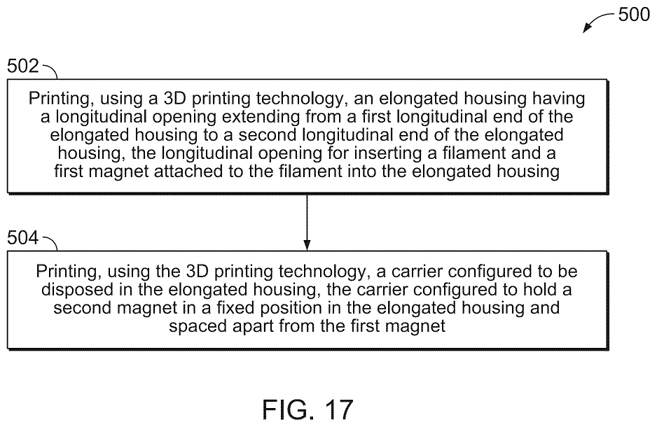

FIG. 17 is an example method 500 for manufacturing the housing 152 or 300 and the first part or carrier 260. At step 502, the method 500 includes printing, using a 3D printing technology, an elongated housing having a longitudinal opening extending from a first longitudinal end of the elongated housing to a second longitudinal end of the elongated housing, the longitudinal opening for inserting a filament and a first magnetic object attached to the filament into the elongated housing. For example, the elongated housing 152 or 300 is printed to include the longitudinal opening 160.

At step 504, the method 500 includes printing, using the 3D printing technology, a carrier configured to be disposed in the elongated housing, the carrier configured to hold a second magnetic object in a fixed position in the elongated housing and spaced apart from the first magnet. For example, the carrier 260 is printed.

In some examples, the 3D printing technology is a 2-photon polymerization technology. A 2-photon polymerization technology can provide precise printing of features with a resolution down to 200 nm and can print the housing 152 or 300 having a diameter of less than 2 mm (e.g., between 0.4 and 1.5 mm) and a length of less than 4 mm (e.g., between 1 and 3 mm).

In some examples, the elongated housing 152 or 300 is printed to include a distance element 164 (e.g., the protrusion or wall) defined by a stepped portion of the sidewall 158 of the elongated housing 300, and the carrier 260 is configured to contact the distance element 164 when inserted into the elongated housing 152.

In some examples, the elongated housing 300 is printed to include a notch element 302 at the second longitudinal end for guiding the filament 200 into the longitudinal opening 160.

A system can include one or more micromechanical resonators 150. Each micromechanical resonator 150 can be configured to have a different resonant frequency. For example, different resonant frequencies can result from different axial lengths of filament 200 being used with each respective housing 152 of the one or more micromechanical resonators 150. For example, different axial lengths of filament 200 between the first end 154 of the housing 152 and the first magnetic object 220 can be used to control the resonant frequency in which the first magnetic object 220 oscillates in response to a magnetic or electromagnetic field (e.g., an external magnetic or electromagnetic field).

The system can include one or more electromagnetic sources external to the one or more micromechanical resonators 150. The one or more electromagnetic sources can provide external magnetic or electromagnetic fields to excite the respective resonant frequencies of the micromechanical resonators 150. For example, the one or more electromagnetic sources can be the electro-magnetic transducers 122 described with reference to FIG. 1 which emit magnetic or electromagnetic fields 166 to excite the respective resonant frequencies of the micromechanical resonators 150.

The system can include one or more sensors external to the one or more micromechanical resonators 150. The one or more sensors can be configured to determine respective positions of the one or more micromechanical resonators 150 based on sensing the respective resonant frequencies. For example, the one or more electromagnetic transducers 122 can be configured in a receive mode in which the one or more electromagnetic transducers 122 sense disturbances in a magnetic or electromagnetic field due to the oscillations of the first magnetic object 220 within the respective micromechanical resonators 150, as described above with reference to FIGS. 3 and 4.

Since each micromechanical resonator 150 has a different resonant frequency, a Fourier transformation of the signal measured by the one or more electromagnetic sources 122 can reveal a spike at the respective resonant frequencies for each micromechanical resonator 150. A computer can use triangulation to determine the coordinates of each micromechanical resonator 150 by processing the portion of the signal associated with each resonant frequency spike of all electromagnetic sources 122. The frequency can be used to separate mechanical resonators and the intensity of the signal sent back from the mechanical resonator to the different coils can be used to determine the position of the mechanical resonators.

The system can include a medical device with the one or more micromechanical resonators 150 disposed in the medical device. In some examples, the micromechanical resonators 150 are embedded in the medical device. For example, the computer is configured to determine a position of the medical device based on sensing the respective resonant frequencies. In some examples, the medical device is a catheter (e.g., catheter 118) and the one or more micromechanical resonators 150 are inside the catheter 118 (e.g., embedded in a sidewall 117 of the catheter 118). In such examples, the one or more micromechanical resonators 150 are allowed to move relative to one another as the catheter 118 bends through a tortuous anatomy of a patient.

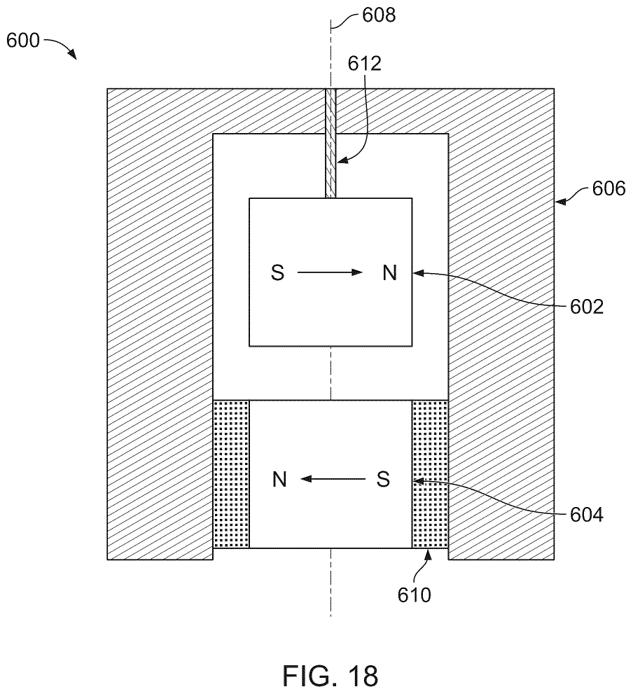

FIG. 18 shows a micromechanical resonator 600 with magnetic objects implemented as cylindrical permanent magnets 602, 604. The micromechanical resonator 600 is substantially similar to micromechanical resonator 150 except that the cylindrical permanent magnets 602, 604 are used, and a carrier 260 may not be needed. The housing 606 may not have a distance element 164 and can instead have a constant inner diameter along the longitudinal axis 608 as shown in FIG. 18. An adhesive 610 can be applied directly between the second magnetic object 604 and the housing 606. The adhesive 610 can be a cyanoacrylate, an epoxy-based adhesive, or a UV curable adhesive. The filament 612 can be the same or similar to the filament 200. When cylindrical permanent magnets 602, 604 are used, the magnetization direction of the cylindrical permanent magnets 602, 604 is perpendicular to the longitudinal axis 608 of the housing as shown in FIG. 18. This is an uncommon polarization direction for cylindrical permanent magnets but helpful if cylindrical magnets are used within a cylindrical housing 606 to utilize the space within the housing 606 efficiently.

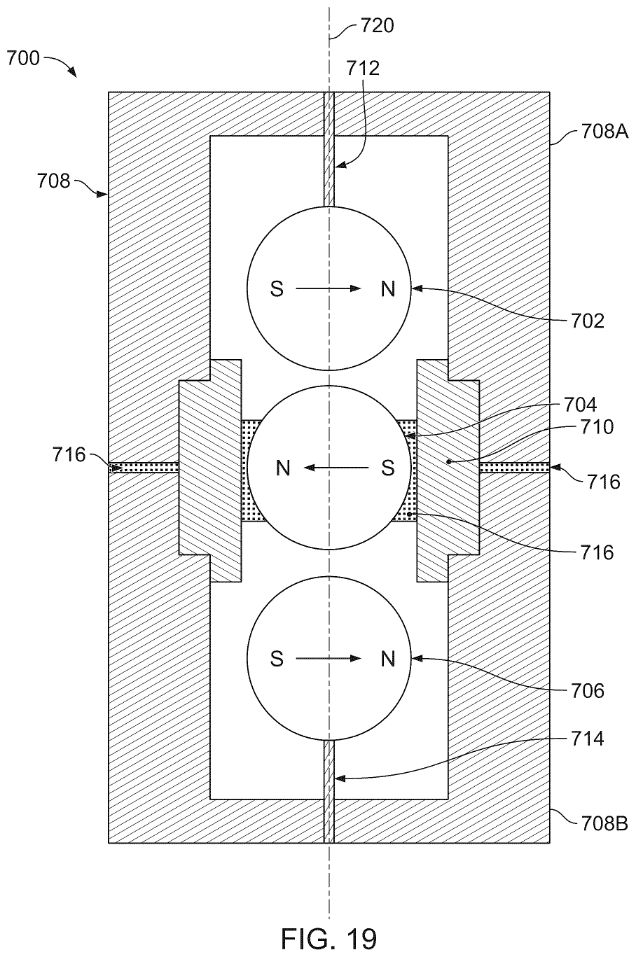

FIG. 19 shows micromechanical resonator 700 with magnetic objects implemented as three spherical permanent magnets 702, 704, 706. The housing 708 is similar to the housing 300 except that it is substantially mirrored about a plane perpendicular to the longitudinal axis 170 that bisects the carrier 260 in the axial direction. The carrier 710 can be inserted between the two housing portions 708A, 708B during assembly to position the second magnetic object 704 within the micromechanical resonator 700. The first magnetic object 702 is attached to a first filament 712 and is substantially similar to the micromechanical resonator 150 described above. The third magnetic object 706 is attached to a second filament 714 at an opposite axial end of the housing 708 (e.g., at an axial center of the second housing portion 708B). The first housing portion 708A has a longitudinal opening (not shown in FIG. 19) that is substantially similar to the longitudinal opening 160 of micromechanical resonator 150, and the second housing portion 708B has a longitudinal opening (not shown in FIG. 19) that is substantially similar to the longitudinal opening 160 of micromechanical resonator 150. Before the housing portions 708A, 708B are attached to one another and adhered (e.g., by the adhesive 716), the first filament 712 with the first magnetic object 702 attached thereto is inserted into the longitudinal opening of the first housing portion 708A, and the second filament 714 with the third magnetic object 706 attached thereto is inserted into the longitudinal opening of the second housing portion 708B. The micromechanical resonator 700 can have a symmetrical setup which can result in the resonator 700 being less sensitive to external fields.

In the three-magnet design shown in FIG. 19, the micromechanical resonator 700 can be tuned to use two resonant frequencies—a first resonant frequency of the oscillation of the first magnetic object 702 about the longitudinal axis 720 and a second resonant frequency of the oscillation of the third magnetic object 706 about the longitudinal axis 720. While the orientation of the micromechanical resonator with two magnetic objects can be determined in six degrees of freedom, three magnetic objects allow for more properties of the mechanical resonator 700 to be determined and/or can provide a redundancy in determined properties. For example, a first resonance can be used to determine a position and/or orientation of the micromechanical resonator 700 while a second resonance can be used to determine a temperature and/or pressure within the mechanical resonator 700. For details about sensing temperature and/or pressure see, e.g., U.S. Pat. No. 11,774,300, which is incorporated herein by reference in its entirety.

In some examples, the first and second magnetic objects 702, 704 can be used to determine a position and orientation of the micromechanical resonator 700 using a first resonance while the second and third magnetic objects 704, 706 can be used to determine the position and orientation of the micromechanical resonator 700 using a second resonant frequency. In some examples, respective positions and/or orientations determined based on each resonant frequency can be compared to one another as a fail-safe redundancy. For example, the position and/or orientation is presumed accurate if both determinations of positions and/or orientations are within a tolerance of one another, and the position and/or orientation is presumed inaccurate if the determinations of positions and/or orientations are not within the tolerance of one another.

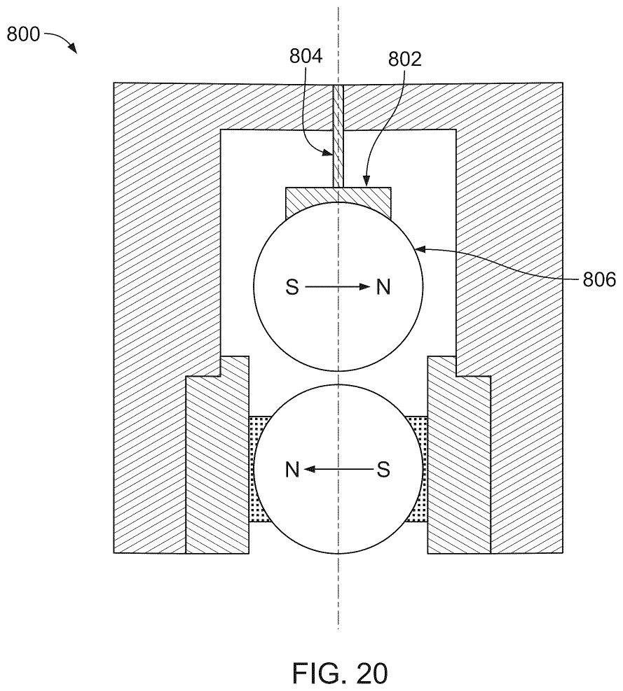

FIG. 20 shows micromechanical resonator 800 with a second part 802 (e.g., an attachment device, a plate, etc.). The micromechanical resonator 800 is substantially the same as micromechanical resonator 150 except for the inclusion of the attachment second part device 802 (e.g., an attachment, a plate, etc.). The second part 802 can help to increase the contact area between the filament 804 and the first magnetic object 806. The filament 804 can be attached to the attachment device 802. In some examples, the filament 804 can be attached to a protrusion of the second device 802 (e.g., an attachment, a plate, etc.). In some examples, the filament 804 can be wound around the second device 802 (e.g., an attachment, a plate, etc.). In some examples, the filament 804 is wound circumferentially around the second device 802 (e.g., an attachment, a plate, etc.). In other examples, the filament 804 is wound axially around the second device 802. In some examples, the filament 804 is wound circumferentially around the second device 802 and axially around the second device 802. The second device 802 can be contoured to match a shape of the first magnetic object 806. An adhesive can be used to adhere the filament 804 to the second device 802 and the second device 802 to the first magnetic object 806, as described above.

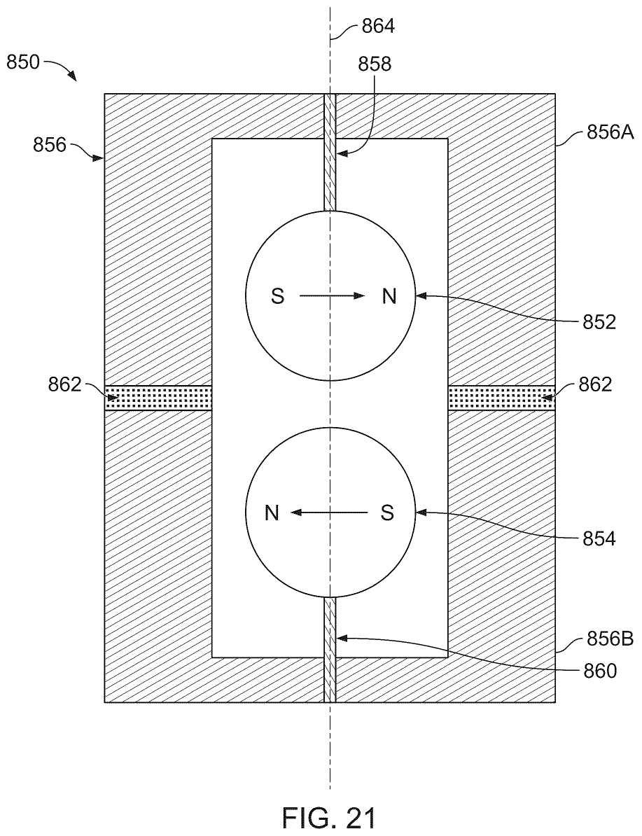

FIG. 21 shows micromechanical resonator 850 with two movable magnetic objects 852, 854. The first magnetic object 852 (e.g., a first spherical permanent magnet, a first cylindrical permanent magnet, a first coil, etc.) is suspended from a first portion 856A of the elongated housing 856 by a first filament 858. In this way, the micromechanical resonator 850 is similar to other micromechanical resonators described herein. However, there is no fixed magnet in the micromechanical resonator 850. Instead, the second magnetic object 854 (e.g., a second spherical permanent magnet, a second cylindrical permanent magnet, a second coil, etc.) is suspended from a second portion 856B of the housing 856 by a second filament 860. The first filament 858 can be attached to the first magnetic object 854 directly (e.g., by adhering the first filament 858 directly to the first magnetic object 852 using an adhesive), or by using an attachment device (such as the attachment device 802). The second filament 860 can be attached to the second magnetic object 854 directly (e.g., by adhering the second filament 860 directly to the second magnetic object 854 using an adhesive), or by using an attachment device (such as the attachment device 802). The first and second magnetic objects 852, 854 have opposite polarities.

When the micromechanical resonator 850 is exposed to a magnetic or electromagnetic field, the first magnetic object 852 oscillates about a longitudinal axis 864 of the housing 856 relative to the second magnetic object 854 which oscillates in an opposite direction about the longitudinal axis 864 of the housing 856. The first and second magnetic objects 852, 854 oscillate relative to the housing 856 without having a fixed magnetic object disposed between the first and second magnetic objects 852, 854. The first and second magnetic objects 852, 854 directly face one another. The micromechanical resonator 850 can have a symmetrical setup which can result in the resonator 850 being less sensitive to external fields.

A number of systems and methods have been described. Nevertheless, it will be understood that various modifications may be made without departing from the spirit and scope of this disclosure.

Some micromechanical resonators can include both spherical and cylindrical permanent magnets. Some micromechanical resonators can include movable permanent magnets as the first magnetic object combined with coils as the second magnetic object. Some micromechanical resonators can include movable coils as the first magnetic object combined with permanent magnets as the second magnetic object. In some examples, the first magnetic object can be a tapered cylindrical permanent magnet with the tapered end away from the second magnetic object.

While the micromechanical resonators have been described with reference to a medical environment of FIG. 1, other applications are possible. For example, using micromechanical resonators to track the 3D position of small animals (e.g., mice, rodents, hamsters, etc.) in a controlled cage (e.g., by attaching the micromechanical resonators in or on the small animals). For example, using micromechanical resonators to track a 3D position of a gesture of a person for consumer gaming or human-machine interface navigation (e.g., by attaching the micromechanical resonators in or on a hand of the person, by attaching the micromechanical resonators in or on a controller to be held by the person, etc.). Other non-exhaustive applications include industrial automation/manufacturing, robotics, skill development/training, military, industrial sensing, etc.

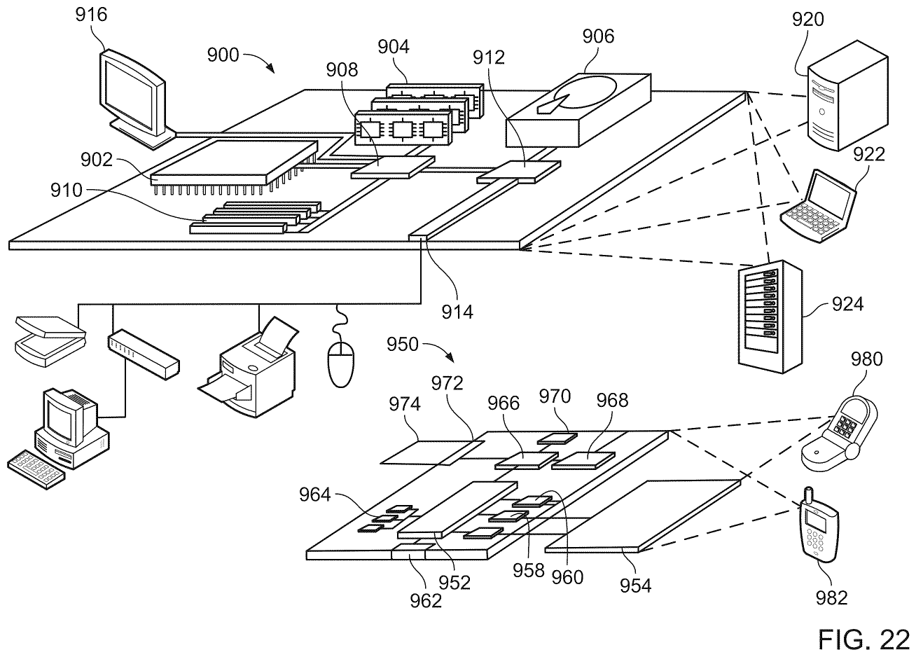

FIG. 22 shows an example computer device 900 and example mobile computer device 950 which can be used to implement the techniques described herein. For example, the computer devices 900, 950 can execute software to determine the position and/or orientation of the micromechanical resonators described herein based on signals received from the one or more electromagnetic sources/receivers 122. As described above, the position and/or orientation of the micromechanical resonators can be used to infer the position and/or orientation of the medical instrument to which the micromechanical resonators are attached (e.g., the catheter 118). The computer devices 900, 950 can then present this information on a display for the medical professional to assist in guiding the medical instrument to the desired location within the anatomy of a patient. In some implementations, the computer system 108 and/or the electromagnetic sources/receivers (e.g., electromagnetic transducer 122) include one or more components of the example computer device 900 and example mobile computer device 950.

Computing device 900 is intended to represent various forms of digital computers, including, e.g., laptops, desktops, workstations, personal digital assistants, servers, blade servers, mainframes, and other appropriate computers. Computing device 954 is intended to represent various forms of mobile devices, including, e.g., personal digital assistants, tablet computing devices, cellular telephones, smartphones, and other similar computing devices. The components shown here, their connections and relationships, and their functions, are meant to be examples only, and are not meant to limit implementations of the techniques described and/or claimed in this document.

Computing device 900 includes processor 902, memory 904, storage device 906, high-speed interface 908 connecting to memory 904 and high-speed expansion ports 910, and low speed interface 912 connecting to low speed bus 914 and storage device 906. Each of components 902, 904, 906, 908, 910, and 912, are interconnected using various busses, and can be mounted on a common motherboard or in other manners as appropriate. Processor 902 can process instructions for execution within computing device 900, including instructions stored in memory 904 or on storage device 906 to display graphical data for a GUI on an external input/output device, including, e.g., display 916 coupled to high speed interface 908. In other implementations, multiple processors and/or multiple busses can be used, as appropriate, along with multiple memories and types of memory. Also, multiple computing devices 900 can be connected, with each device providing portions of the necessary operations (e.g., as a server bank, a group of blade servers, or a multi-processor system).

Memory 904 stores data within computing device 900. In one implementation, memory 904 is a volatile memory unit or units. In another implementation, memory 904 is a non-volatile memory unit or units. Memory 904 also can be another form of computer-readable medium (e.g., a magnetic or optical disk). Memory 904 may be non-transitory.

Storage device 906 is capable of providing mass storage for computing device 900. In one implementation, storage device 906 can be or contain a computer-readable medium (e.g., a floppy disk device, a hard disk device, an optical disk device, a tape device, a flash memory, or other similar solid state memory device, or an array of devices, such as devices in a storage area network or other configurations.) A computer program product can be tangibly embodied in a data carrier. The computer program product also can contain instructions that, when executed, perform one or more methods (e.g., those described above.) The data carrier is a computer-or machine-readable medium, (e.g., memory 904, storage device 906, memory on processor 902, and the like.)

High-speed controller 908 manages bandwidth-intensive operations for computing device 900, while low speed controller 912 manages lower bandwidth-intensive operations. Such allocation of functions is an example only. In one implementation, high-speed controller 908 is coupled to memory 904, display 916 (e.g., through a graphics processor or accelerator), and to high-speed expansion ports 910, which can accept various expansion cards (not shown). In the implementation, low-speed controller 912 is coupled to storage device 906 and low-speed expansion port 914. The low-speed expansion port, which can include various communication ports (e.g., USB, Bluetooth®, Ethernet, wireless Ethernet), can be coupled to one or more input/output devices, (e.g., a keyboard, a pointing device, a scanner, or a networking device including a switch or router, e.g., through a network adapter.)

Computing device 900 can be implemented in a number of different forms, as shown in the FIG. 22. For example, it can be implemented as standard server 920, or multiple times in a group of such servers. It also can be implemented as part of rack server system 924. In addition or as an alternative, it can be implemented in a personal computer (e.g., laptop computer 922.) In some examples, components from computing device 900 can be combined with other components in a mobile device (not shown), e.g., device 954. Each of such devices can contain one or more of computing device 900, 954, and an entire system can be made up of multiple computing devices 900, 954 communicating with each other.

Computing device 954 includes processor 952, memory 964, an input/output device (e.g., display 954, communication interface 966, and transceiver 968) among other components. Device 954 also can be provided with a storage device, (e.g., a microdrive or other device) to provide additional storage. Each of components 954, 952, 964, 954, 966, and 968, are interconnected using various buses, and several of the components can be mounted on a common motherboard or in other manners as appropriate.

Processor 952 can execute instructions within computing device 954, including instructions stored in memory 964. The processor can be implemented as a chipset of chips that include separate and multiple analog and digital processors. The processor can provide, for example, for coordination of the other components of device 954, e.g., control of user interfaces, applications run by device 954, and wireless communication by device 954.

Processor 952 can communicate with a user through control interface 958 and display interface 956 coupled to display 954. Display 954 can be, for example, a TFT LCD (Thin-Film-Transistor Liquid Crystal Display) or an OLED (Organic Light Emitting Diode) display, or other appropriate display technology. Display interface 956 can comprise appropriate circuitry for driving display 954 to present graphical and other data to a user. Control interface 958 can receive commands from a user and convert them for submission to processor 952. In addition, external interface 962 can communicate with processor 942, so as to enable near area communication of device 954 with other devices. External interface 962 can provide, for example, for wired communication in some implementations, or for wireless communication in other implementations, and multiple interfaces also can be used.

Memory 964 stores data within computing device 954. Memory 964 can be implemented as one or more of a computer-readable medium or media, a volatile memory unit or units, or a non-volatile memory unit or units. Expansion memory 974 also can be provided and connected to device 954 through expansion interface 972, which can include, for example, a SIMM (Single In Line Memory Module) card interface. Such expansion memory 974 can provide extra storage space for device 954, or also can store applications or other data for device 954. Specifically, expansion memory 974 can include instructions to carry out or supplement the processes described above, and can include secure data also. Thus, for example, expansion memory 974 can be provided as a security module for device 954, and can be programmed with instructions that permit secure use of device 954. In addition, secure applications can be provided through the SIMM cards, along with additional data, (e.g., placing identifying data on the SIMM card in a non-hackable manner.)

The memory can include, for example, flash memory and/or NVRAM memory, as discussed below. In one implementation, a computer program product is tangibly embodied in a data carrier. The computer program product contains instructions that, when executed, perform one or more methods, e.g., those described above. The data carrier is a computer-or machine-readable medium (e.g., memory 964, expansion memory 974, and/or memory on processor 952), which can be received, for example, over transceiver 968 or external interface 962.

Device 954 can communicate wirelessly through communication interface 966, which can include digital signal processing circuitry where necessary. Communication interface 966 can provide for communications under various modes or protocols (e.g., GSM voice calls, SMS, EMS, or MMS messaging, CDMA, TDMA, PDC, WCDMA, CDMA2000, or GPRS, among others.) Such communication can occur, for example, through radio-frequency transceiver 968. In addition, short-range communication can occur, e.g., using a Bluetooth®, WiFi, or other such transceiver (not shown). In addition, GPS (Global Positioning System) receiver module 970 can provide additional navigation-and location-related wireless data to device 954, which can be used as appropriate by applications running on device 954. Sensors and modules such as cameras, microphones, compasses, accelerators (for orientation sensing), etc. may be included in the device.

Device 954 also can communicate audibly using audio codec 960, which can receive spoken data from a user and convert it to usable digital data. Audio codec 960 can likewise generate audible sound for a user, (e.g., through a speaker in a handset of device 954.) Such sound can include sound from voice telephone calls, can include recorded sound (e.g., voice messages, music files, and the like) and also can include sound generated by applications operating on device 954.

Computing device 954 can be implemented in a number of different forms, as shown in the FIG. 22. For example, it can be implemented as cellular telephone 980. It also can be implemented as part of smartphone 982, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor. The programmable processor can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a device for displaying data to the user (e.g., a CRT (cathode ray tube) or LCD (liquid crystal display) monitor), and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such back end, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Accordingly, other implementations are within the scope of the following claims.

1. A device comprising:
an elongated housing having a first end, a second end, and a sidewall extending from the first end to the second end, a longitudinal opening extending through the sidewall of the elongated housing and extending from an axial center at the first end of the elongated housing to the second end of the elongated housing;
a filament disposed in the elongated housing with a first end of the filament attached to the first end of the elongated housing at the axial center;
a first magnetic object disposed in the elongated housing with a second end of the filament attached to the first magnetic object; and
a second magnetic object disposed in the elongated housing and being spaced apart from the first magnetic object,
wherein the filament is configured to allow the first magnetic object to oscillate about a longitudinal axis of the elongated housing relative to the second magnetic object in response to a magnetic or electromagnetic field.

2. The device of claim 1, wherein the longitudinal opening is configured to allow the filament to be inserted into the elongated housing with the second end of the filament attached to the first magnetic object prior to the first end of the filament being attached and fixed to the first end of the elongated housing at the axial center.

3. The device of claim 1, wherein the longitudinal opening can be closed with the filament inside the elongated housing and the second end of the filament attached to the first magnetic object.

4. The device of claim 1, wherein a magnetic axis of the first magnetic object is perpendicular to the longitudinal axis of the elongated housing, a magnetic axis of the second magnetic object is perpendicular to the longitudinal axis of the elongated housing, and the magnetic axis of the first magnetic object and the magnetic axis of the second magnetic object have opposite polarities.

5. The device of claim 1, wherein the first magnetic object is configured to oscillate about the longitudinal axis of the elongated housing relative to the second magnetic object while maintaining a fixed axial distance to the second magnetic object in response to the magnetic or electromagnetic field.

6. The device of claim 1, wherein the first magnetic object is configured to be rotated out of an equilibrium orientation in response to the magnetic or electromagnetic field to initiate the oscillation relative to the second magnetic object.

7. The device of claim 1, wherein the first magnetic object is suspended in the elongated housing by the filament and does not contact the sidewall of the elongated housing.

8. The device of claim 1, comprising a carrier disposed in the elongated housing, wherein the second magnetic object is fixed in the carrier, and the carrier positions the second magnetic object at a pre-determined distance from the first magnetic object.

9. The device of claim 8, wherein the sidewall of the elongated housing comprises a distance element configured to contact the carrier to position the second magnetic object at the pre-determined distance from the first magnetic object, the distance element having a curved inner rim.

10. The device of claim 1, wherein the second end of the filament is adhered to the first magnetic object by a cyanoacrylate, an epoxy-based adhesive, or a UV curable adhesive.

11. The device of claim 10, wherein the first end of the filament is adhered to the first end of the elongated housing at the axial center by the cyanoacrylate, the epoxy-based adhesive, or the UV curable adhesive.

12. The device of claim 1, comprising a second part, wherein the second end of the filament is directly attached to the second part, and the second part is directly attached to the first magnetic object.

13. The device of claim 1, wherein the second magnetic object is fixed relative to the elongated housing.

14. The device of claim 1, wherein the filament is a first filament, and the device comprises a second filament attached to the second magnetic object, wherein the second filament is configured to allow the second magnetic object to oscillate about the longitudinal axis of the elongated housing relative to the first magnetic object in response to the magnetic or electromagnetic field.

15. The device of claim 14, comprising a third magnetic object disposed between the first and second magnetic objects, wherein the third magnetic object is fixed to the elongated housing.

16. The device of claim 1, wherein the filament comprises a Kevlar® fiber, an ultra-high density polyethylene fiber, or a carbon fiber.

17. The device of claim 1, wherein a torsional stiffness of the filament is less than 1e-7 Nm/rad, and an axial stiffness of the filament is greater than 1 GPa.

18. The device of claim 1, wherein the first magnetic object is a first permanent magnet, and the second magnetic object is a second permanent magnet.

19. The device of claim 1, wherein the device does not require electronics to produce the oscillations.

20. A device comprising:
an elongated housing comprising a longitudinally-extending opening;
a filament having a first end fixed to the elongated housing within the longitudinally-extending opening;
a first magnetic object disposed in the elongated housing with a second end of the filament fixed to the first magnetic object; and
a second magnetic object disposed in the elongated housing and being spaced apart from the first magnetic object, wherein the filament is configured to allow the first magnetic object to oscillate about a longitudinal axis of the elongated housing relative to the second magnetic object.

21-53. (canceled)
